(19) United States
(12) Patent Application Publication
(10) Pub. No.: US 2015/0359483 A1
(43) Pub. Date: Dec. 17, 2015

(54) METHODS AND SYSTEMS FOR IMPROVING PERCEIVED AGE BASED ON PHENOTYPIC AND GENETIC FEATURES OF THE SKIN

(71) Applicant: Genocosmetics Lab SL, Barcelona (ES)

(72) Inventors: Miguel Angel Herranz Rosa, Barcelona (ES); Vicente Carles Alonso Usero, Betera (ES); Jordi Naval Chamosa, Barcelona (ES)

(73) Assignee: Genocosmetics Lab SL, Barcelona (ES)

(21) Appl. No.: 14/484,975

(22) Filed: Sep. 12, 2014

Related U.S. Application Data
(60) Provisional application No. 61/877,653, filed on Sep. 13, 2013.

(51) Int. Cl. A61B 5/00; C12Q 1/68

(52) U.S. Cl. CPC A61B 5/4848; A61B 5/441; A61B 5/7264; C12Q 1/6883; C12Q 2600/156; C12Q 2600/106

(57) ABSTRACT

The present invention relates to methods and systems for improving perceived age of an individual. Particularly, the present invention relates to methods and systems by which phenotypic features and certain polymorphisms in one or more genes of an individual can be measured and classified to evaluate the perceived age of an individual. The invention also relates to methods useful to define therapies (products or treatments) to improve perceived age of an individual, to evaluate the efficacy of therapies and to benchmark therapies in order to determine its market value and customer claims.

### CROSS REFERENCE TO RELATED APPLICATIONS

This application is a non-provisional application claiming priority to U.S. Provisional Application Ser. No. 61/877,653, filed Sep. 13, 2013, the entire content of which is hereby incorporated by reference.

### BACKGROUND OF THE INVENTION

Biological age of a subject is person's chronological age, whereas perceived age is defined as the age that a person is visually estimated to have, based on their physical appearance. Biological age and perceived age are generally measured in years and parts thereof.

Some individuals show a difference between their biological age and their perceived age. The difference between the biological age and perceived age can be a result of various intrinsic and extrinsic factors including but not limited to, exposure to sunlight, pollution, nicotine, and diet or sleeping habits.

Research on same-sexed twins suggests an important genetic contribution to perceived age [1, 2], of around 60%. Thus, the remaining 40% of the variation in perceived age is due to non-genetic factors. Among them, smoking and sun exposure are the environmental factors of major importance for premature skin wrinkling or facial ageing [3].

Perceived age, which is widely used by clinicians in a non-structured, non-standardized measurement, as a general indicator of patient's health is a robust biomarker of ageing that predicts survival in certain groups of patients, and that correlates with important functional and molecular ageing phenotypes [4]. Besides patient survival, perceived age correlates with age-related phenotypes such as physical and cognitive functioning and leucocyte telomere length.

The cosmetic industry uses perceived age assessment to determine the efficacy of treatments as per example to quantify the efficacy of multisyringe hyaluronic acid treatment [5] or plastic surgery [6],

Perceived age is measured by clinical assessment [7] [8], Therefore, there is a need for an objective method to determine the perceive age of a person in a faster, better, and more accurate manner.

A method for diagnosing person's perceived age could be applied as an easy and non-invasive method to diagnosis person's health such as waist circumference method as a marker of metabolic syndrome [9] or cardiovascular events [10] and for the evaluation of the efficacy of cosmetic treatments such as contact thermography, morphometric measures of thigh circumference, and microcirculation evaluation used in cellulites [11].

The present invention is directed towards a new, non-obvious and more accurate method for improving perceived age by which phenotypic features of individuals, and certain polymorphism in one or more genes can be measured and classified to evaluate and improve the perceived age of an individual.

The method of the present invention is a consistent and standardized method for improving person's perceived age that allows the measurement of a person's perceived age over time and the validation of their treatments which include but is not limited to cosmetic treatment, exercise, nutritional complements, diets alternative medicine such as yoga, meditation, relaxation, pilates, laughter therapy, personal growth therapy, psychotherapy, nutritional complements, diets and the similar.

### SUMMARY OF THE INVENTION

The present invention relates to methods and systems for improving perceived age of an individual. Particularly, the present invention relates to methods and systems by which phenotypic features and certain polymorphisms in one or more genes of an individual can be measured and classified to evaluate the perceived age of an individual. The invention also relates to methods useful to define products or treatments to improve perceived age of an individual, to evaluate the efficacy of products or treatments, and to benchmark products or treatments in order to determine its market value and customer claims.

The method disclosed in the invention comprises a set of algorithms to improve person's perceived age. Particularly, a first algorithm 1 for diagnosing perceived age based on an ensemble of phenotypic features of the skin, a second algorithm 2 for classifying individuals based on an ensemble of multiple polymorphisms associated to certain properties of the skin, and a third algorithm 3 to design therapies improve person's perceived age based on an ensemble of phenotypic and genotypic features of the skin (FIG. 1).

The algorithm 1 disclosed in the invention simulates and improves the behavior of clinical assessment performed by an expert panel, determining a person's perceived age with the validated facial grading scale based on individual phenotype criteria given by A. Carruthers [12].

The algorithm 2 disclosed in the invention classifies individuals into clusters defined as genotypic variables, by evaluating the occurrence in the human's genome of polymorphisms in one or more genes associated with certain properties of the skin that contribute to person's perceived age.

The algorithm 3 disclosed in the invention uses certain polymorphisms in one or more genes of an individual, in combination with certain physical properties of the skin, particularly its elasticity, hydration and antioxidant capacity to design methods and therapies to evaluate and improve the perceived age of individuals.

The algorithm of the present invention provides an objective quantification of perceived age, which can be used as a measure to evaluate the overall health of an individual, including but not limited to, skin aging or ageing related disorders associated with the key organs such as liver, lungs, kidney, heart, skin, muscles or bones and biological systems like the central nervous system, digestive, reproduction system and the similar, and more particularly, skin aging.

The invention also provides a diagnostic method based on determining a person's perceived age, useful to design an optimal treatment to improve person's perceived age.

The invention also relates to a consistent and standardized diagnostic method that allows measurement of a perceived age over time, thus allowing validation of treatment which include but not limited to cosmetic treatment, exercise, nutritional complements, diets alternative medicine such as yoga, meditation, relaxation, pilates, laughter therapy, personal growth therapy, psychotherapy, and the similar.

The invention also relates to a method which determines a person's perceived age, useful to benchmark the product in order to determine its market value and customer claims.

### BRIEF DESCRIPTION OF THE DRAWINGS

FIG. 1 Schematical representation of the methods and systems for improving perceived age of an individual.

FIG. 2 Distribution of a cohort of 120 female volunteers into 10 clusters defined as genetic variables by the algorithm.

FIG. 3 Schematical representation of the result of the nonlinear dimensionality reduction method. The phenotype of an individual corresponds to a definition of 22 variables, which in mathematical terms means a vector of dimension 22, by using nonlinear dimensionality reduction methods, and neuronal network this space is reduced to two dimensions known as x-genos and y-genos.

### DETAILED DESCRIPTION OF THE INVENTION

A set of phrases and words to be used in this document are defined in order to avoid uncertainty about the terms.

Biological age: We define the biological age of a subject as a person's chronological age

Perceived age: We define the perceived age of a subject as the age that a person is visually estimated to have based on their physical appearance.

Biological age and perceived age are generally measured in years and parts thereof. We define the perceived age of a subject as an integer in the range of [−5, +5] years defined by a committee of experts by applying the validated facial grading given by A. Carruthers age [12].

Phenotype: We define phenotype as the set of measurements observed on a subject in terms of its physical appearance. Phenotypes arise from a combination of genetic background and environmental variables.

Phenotypic variable or phenotypic parameter is a measurement observed on a subject in terms of their physical appearance.

Genotype: We define genotype as a set of polymorphism of one or more genes evaluated in a subject in terms of their genetic background.

Genetic polymorphism: We define a genetic polymorphism as the presence of two or more distinct phenotypes in a population due to the expression of different alleles of a given gene.

Single Nucleotide Polymorphism (SNP): We define a SNP as a genetic polymorphism between two genomes that is based on deletion, insertion, or exchange of a single nucleotide.

Genotypic variable or cluster is a set of one or more polymorphisms associated with one or more genes related to certain properties of the skin (phenotypes) that contributes to perceived age of an individual.

Therapies of this invention relates to cosmetic treatments, alternative medicines or other methods of improving the health and appearance of a human being.

Cosmetic treatment of this invention means a cosmetic products or treatments of biological interest include but not limited to products that have an moisturizing effect, anti-aging, structuring effect, increasing the brightness, thickness and microcirculation, wrinkle fillers, balance the homeostasis of the skin, skin regeneration, dermal metabolism stimulation, skin repair, protection against environmental contamination, revitalizing, improving ionic equilibrium of the skin, energizing for tired skin, balance the pH of the skin, anti-irritation, decreased skin sensitivity, softness, skin conditioning, lifting effect, increases elasticity and firmness, improve eye contour skin barrier, acne redactor, inhibit melanin synthesis and the similar.

Alternative medicine of this invention means treatments of biological interest include but not limited to yoga, pilates, meditation, relaxation, laughter therapy, personal growth therapy, psychotherapy, nutritional complements, exercise, ayurvedic medicine, traditional Chinese medicine, homeopathy, naturopathy, energy therapies, biofields, electromagnetic fields, mind body therapies, massage, chiropractic, osteopathy and the similar.

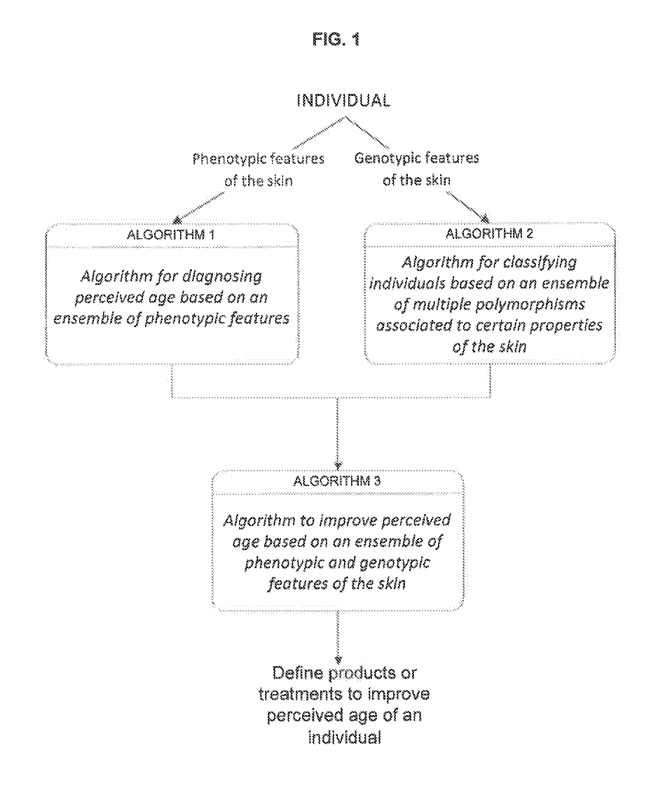

The method disclosed in the invention comprises a set of algorithms to improve person's perceived age. Particularly, algorithm 1 for diagnosing perceived age based on an ensemble of phenotypic features of the skin, algorithm 2 for classifying individuals based on an ensemble of multiple polymorphisms associated to certain properties of the skin, and algorithm 3 to improve person's perceived age based on an ensemble of phenotypic and genotypic features of the skin (FIG. 1).

The method of the present invention comprises the steps of:
a. Measuring phenotypic features of the skin that contributes to person's perceived age
b. Measuring one or multiple polymorphisms in one or more genes associated with certain properties of the skin that contributes to perceived age
c. Inputting the phenotypic data into a computerized knowledge system that contains a novel algorithm that predicts perceived age of an individual
d. Inputting the genotypic data into a computerized knowledge system that contains a novel algorithm that classify individuals into clusters defined as genotypic variables, associated to certain properties of the skin that contribute to person's perceived age.
e. Use a third algorithm to define products or treatments to improve perceived age of an individual, to evaluate the efficacy of products or treatments, and to benchmark products or treatments in order to determine its market value and customer claims.
f. Wherein the algorithm is derived from an observed correlation between genotypic and physical variables and perceived age, and uses a combination of the genotypic and physical variables to predict and improve the perceived age of individuals

### Variables Used to Evaluate the Contribution of Phenotypic Features of the Skin to Perceived Age

A set of parameters known as phenotypic features are defined to develop the algorithm. Two sets of data of phenotypic features of the skin are collected by measuring (1) biophysical parameters derived from ANTERA 3D Miravex device (Dublin, Ireland) or the similar and (2) clinical assessment by an expert committee.

Biophysical parameters derived from ANTERA 3D Miravex device include but not limited to wrinkles and roughness, number of wrinkles, depth of wrinkles, pigmentation, concentration of melanin, distribution (heterogeneity) of melanin, superficial vascular component, concentration of hemoglobin, distribution (heterogeneity) of hemoglobin, facial furrows, nasogenian furrow, labiomental groove, roughness and the similar.

In a particular embodiment, the estimation of the concentration of melanin was assessed in the jaw-cheek area of the face by using the biophysical parameters; a) the concentration of melanin, b) the index of variation-heterogeneity of melanin distribution, c) the relative percentage variation and d) the distribution method of the melanin in the area of study in the face.

In a particular embodiment, the estimation of the concentration of hemoglobin was assessed in the jaw-cheek area of the face by using the biophysical parameters; a) the average value of hemoglobin, b) the index of variation-heterogeneity of hemoglobin distribution, c) the relative percentage variation, and d) the distribution method of the hemoglobin in the area of study in the face.

In a particular embodiment, the roughness index of the skin was assessed to estimate the number and intensity of wrinkles by measuring the length and depth of wrinkles in nasogenian furrows and the labiomental grooves area.

Clinically assessed parameters are included but not limited to the position of eyebrows, state of periorbital wrinkles, state of facial wrinkles, evaluation of the labiomental groove and the similar.

In a preferred embodiment, the position of eyebrows is evaluated and scored from 0 (youthful and fresh look, and arched eyebrows) to 4 (droopy and almost flat eyebrows with visible folds and tired appearance).

In a preferred embodiment, the forehead wrinkles is quantified in both resting and dynamic position (maximum elevation of the forehead) in the right and left part of the forehead. The forehead wrinkles are scored from 0 (no wrinkles) to 4 (severe wrinkles).

In a preferred embodiment, the labiomental grooves (“Puppet wrinkles”) are evaluated and scored from 0 (no visible folds) to 4 (extremely long and deep folds).

In a preferred embodiment, the periorbital wrinkles (“Crow's feet”) are evaluated at rest and in movement (maximum contraction of the orbicular muscle) in the right and left periorbital area. The periorbital wrinkles are scored from 0 (none) to 4 (severe).

### Variables Used to Evaluate the Contribution of Genotypic Features of the Skin to Perceived Age

A set of genotypic variables, known as one or multiple polymorphisms in one or more genes associated with certain properties of the skin that contribute to perceived age, is defined to develop the algorithm.

The invention relates to the methods and systems for improving perceived age in an individual by evaluating the occurrence in the human's genome of genetic polymorphisms that are associated with disorders, including any type of disorder, regardless if it is a skin disorder or not.

The method involves determining whether one or multiple polymorphisms associated with certain properties of the skin that contribute to perceived age, occur in the genome of human's subjects of the study.

Particularly, the method comprises measuring occurrence in the human's genome of polymorphisms in genes associated with certain properties of the skin that contributes to perceived age selected from the group consisting of

genes which encode a protein involved in skin hydration

genes which encode a protein involved in skin elasticity

genes which encode a protein involved in antioxidant capacity of the skin

More particularly, the method comprises measuring occurrence in the human's genome of polymorphisms in one or more genes associated with certain properties of the skin that contribute to perceived age selected from the group consisting of

genes that encode matrix metalloproteinase-1 protein

genes that encode matrix metalloproteinase-3 protein

genes that encode matrix metalloproteinase-9 protein

genes that encode interleukin-6 protein

genes that encode aquaporin-3 protein

genes that encode NAD(P)H dehydrogenase [quinone] 1 protein

genes that encode superoxide dismutase [Mn], mitochondrial protein

genes that encode nuclear factor erythroid 2-related factor 2 protein

genes that encode glutathione peroxidase 1 protein

genes that encode catalase protein

In a preferred embodiment, the method comprises measuring occurrence in the human's genome of polymorphism in one or more genes associated with certain properties of the skin that contribute to perceived age, wherein the polymorphism is selected from the group consisting of rs1799750, rs3025058, rs1800795, rs3918242, rs17553719, rs1800566, rs4880, rs1141718, rs35652124, rs6706649, rs6721961, rs1050450, rs1001179 and combinations thereof.

### Algorithm 1: Algorithm for Diagnosing Perceived Age Based on an Ensemble of Phenotypic Features of the Skin

Data on the perceived age of subjects measured by the test of A. Carruthers [12] is taken in several clinical assessments conducted by one expert person or by an expert committee.

In a preferred embodiment, a set of photos of an individual is taken in a standardized manner. Photographs are assessed blindly and independently by a committee of experts who assign the age attributed to each one of the subjects based on their expert opinion and professional experience as well as, by using the validated scale age of the facial age gradation suggested by A. Carruthers [12],

In a preferred embodiment, perceived age of a person is defined as an integer in the range of [−5, +5] years defined by a committee of experts upon applying the validated facial grading scale given by A. Carruthers [12]. As per example, +2 indicates that the subject appears to have two years more than their actual age, and −3 indicates that the subject has the perceived age equivalent of 3 years younger than their actual age.

The algorithm 1 of the present invention takes phenotype data as variables and expert assignments as objective data.

The algorithm is based on Artificial Neural Networks. The system developed learns the relations between biophysical and clinically assessed parameters of subjects by using supervised mathematical learning techniques.

The phenotype of an individual corresponds to a definition of 22 variables, which in mathematical terms means a vector of dimension 22. The disclosed algorithm uses nonlinear dimensionality reduction methods by means of supervised neural networks to reduce the number of phenotypic variables to two variables, and particularly Sammon mapping [14] and Artificial Neural Network, back propagation technics [13].

More particularly, the 22 original dimensions are reduced to two new dimensions identified herein as x-genos and y-genos.

By using phenotypic features, the algorithm 1 of the invention was able to determine the perceived age of a customer from their phenotypic data with an accuracy of 92%.

### Algorithm 2: Algorithm for Classifying Individuals Based on an Ensemble of Multiple Polymorphism Associated to Certain Properties of the Skin

The method classifies individuals into clusters defined as genotypic variables, by evaluating the occurrence in the human's genome of polymorphisms in one or more genes associated with certain properties of the skin that contribute to person's perceived age.

Clusters were defined on the basis of two principles, (1) equiprobability which means allowing one to assign equal probabilities to outcomes when they are considered as equipossible or to be “equally alike” in some sense and (2) control of homogeneity which means that the characteristics of each cluster (genotypic variable) should be homogeneous within the group and, as much as possible differential between groups and it should be maximized from a genetic point of view.

Particularly, the method comprises assessing occurrence in the human's genome of polymorphism in one or more genes associated with certain properties of the skin that contributes to perceived age, wherein the polymorphism was selected from the group consisting of rs1799750, rs3025058, rs1800795, rs3918242, rs17553719, rs1800566, rs4880, rs1141718, rs35652124, rs6706649, rs6721961, rs1050450, rs1001179 and combinations thereof.

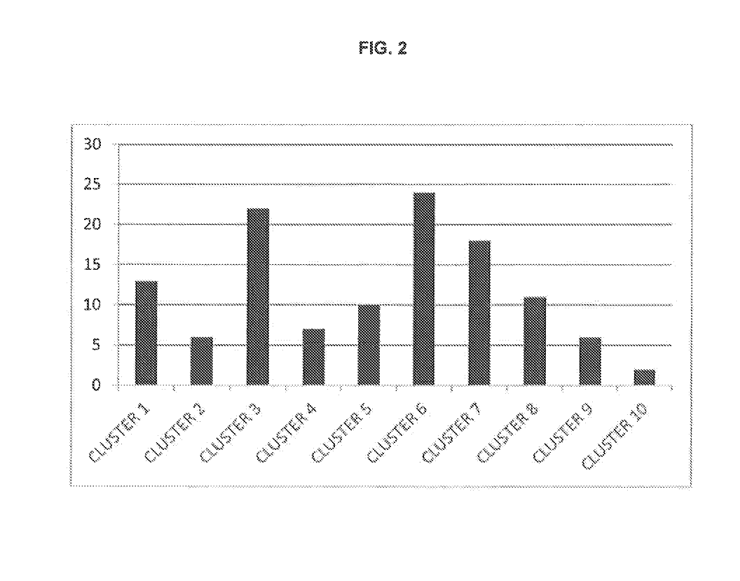

More particularly, the algorithm classifies individuals into 10 clusters defined as genotypic variables by evaluating the occurrence in the human's genome of polymorphism in one or more genes associated with certain properties of the skin that contribute to perceived age, by using grouping or clustering techniques, and more particularly Kmeans technique [13] (FIG. 2).

The algorithm calculates the contribution of the set of polymorphisms that define a genetic variable (Table 1)

| | Relevance of a polymorphism in each genetic variable (cluster) | | | | | | | | | |
|---|---|---|---|---|---|---|---|---|---|---|
| Polymorphism | 1 | 2 | 3 | 4 | 5 | 6 | 7 | 8 | 9 | 10 |
| rs1800566 | 50% | 75% | 53% | 16% | 29% | 45% | 53% | 78% | 100% | 100% |
| rs1799750 | 38% | 33% | 32% | 32% | 42% | 24% | 25% | 28% | 15% | 100% |
| rs4880 | 15% | 29% | 79% | 83% | 19% | 76% | 100% | 48% | 21% | 100% |
| rs1141718 | 73% | 100% | 100% | 100% | 100% | 100% | 100% | 100% | 100% | 100% |
| rs35652124 | 47% | 76% | 75% | 65% | 75% | 75% | 75% | 76% | 100% | 100% |
| rs6706649 | 20% | 44% | 100% | 100% | 31% | 44% | 66% | 76% | 50% | 100% |
| rs6721961 | 46% | 100% | 51% | 18% | 31% | 27% | 32% | 57% | 100% | 50% |
| rs1050450 | 35% | 76% | 42% | 75% | 40% | 32% | 41% | 78% | 76% | 100% |
| rs1001179 | 18% | 76% | 44% | 16% | 41% | 43% | 52% | 75% | 27% | 100% |
| rs3025058 | 77% | 81% | 27% | 100% | 100% | 76% | 45% | 22% | 81% | 100% |
| rs1800795 | 73% | 25% | 75% | 26% | 76% | 55% | 24% | 75% | 81% | 100% |
| rs17553719 | 50% | 100% | 100% | 32% | 27% | 49% | 26% | 28% | 44% | 75% |
| rs3918242 | 77% | 44% | 100% | 100% | 85% | 44% | 100% | 100% | 100% | 100% |

The effect of the set of polymorphisms that define a genetic variable on the expression and/or activity of proteins that confers certain properties of the skin involved on person's perceived age is calculated. Particularly, (1) indicates if the variant increases the expression or the activity of the protein encoded by the gene and (−1) indicates if the variant decreases the expression or the activity of the protein encoded by the gene (Table 2).

TABLE 2
Effect of the most relevance polymorphism on protein activity or
expression

| | Effect of a polymorphism in each | | | | | | | | | |
|---|---|---|---|---|---|---|---|---|---|---|
| | genetic variable (cluster) | | | | | | | | | |
| Polymorphism | 1 | 2 | 3 | 4 | 5 | 6 | 7 | 8 | 9 | 10 |
| rs1800566 | | −1 | | | | | | −1 | −1 | −1 |
| rs1799750 | | | | | | | | | | −1 |
| rs4880 | | | | | | −1 | 1 | | | 1 |
| rs1141718 | | | | | | | | | | |
| rs35652124 | | −1 | −1 | −1 | −1 | −1 | −1 | −1 | 1 | 1 |
| rs6706649 | | | 1 | 1 | | | 1 | −1 | | 1 |
| rs6721961 | | 1 | | | | | | | 1 | |
| rs1050450 | | −1 | | −1 | | | | −1 | −1 | −1 |
| rs1001179 | | −1 | | | | | | −1 | | 1 |
| rs3025058 | −1 | | | 1 | 1 | −1 | | | | 1 |
| rs1800795 | | | −1 | | −1 | | | −1 | | −1 |
| rs17553719 | | 1 | 1 | | | | | | | −1 |
| rs3918242 | −1 | | 1 | 1 | | | 1 | 1 | 1 | −1 |

The overall contribution of the set of polymorphisms to skin properties involved on person's perceived age is calculated. Particularly, (+) indicates if the genotypic variant has a positive effect on a certain skin property or (−) if the genetic variant has a negative effect on a certain skin property (Table 3).

TABLE 3
Overall contribution of the set of polymorphisms that define a
genetic variable on the main skin properties involved
in person's perceived age.

| Clusters | Skin Properties | | |
|---|---|---|---|
| (Genotypic | Antioxidant | Skin | Skin |
| variables) | Capacity | Elasticity | Hydration |
| 1 | 0 | −2 | 0 |
| 2 | −3 | 0 | 1 |
| 3 | 0 | 0 | 1 |
| 4 | −1 | 2 | 0 |
| 5 | −1 | 0 | 0 |
| 6 | −1 | −1 | 0 |
| 7 | −1 | 1 | 0 |
| 8 | −6 | 1 | 0 |
| 9 | 1 | 1 | 0 |
| 10 | 3 | −3 | −1 |

In a preferred embodiment, the method classifies individuals by evaluating the occurrence in the human's genome of polymorphisms described herein, into 10 clusters defined as genotypic variables consisting of
Cluster 1_genetic susceptibility to loss of elasticity of the skin due to polymorphisms in genes that encode matrix metalloproteinase-3 and matrix metalloproteinase-9, and more particularly individuals with polymorphism rs3025058 and rs3918242.
Cluster 2_genetic susceptibility to oxidative damage mainly due to polymorphism in genes that encode NAD(P)H dehydrogenase [quinone] 1, nuclear factor erythroid 2-related factor 2, glutathione peroxidase 1 and catalase, and more particularly individuals with polymorphism rs1800566, rs35652124, rs6721961, rs1050450 and rs1001179, together with a genetic susceptibility to maintain the appropriate hydration levels of the skin due to polymorphism in genes that encodes aquaporin-3, and more particularly the polymorphism rs17553719.
Cluster 3_genetic susceptibility to maintain the appropriate hydration levels of the skin due to polymorphism in genes that encode aquaporin-3, and more particularly the polymorphism rs17553719.
Cluster 4_genetic susceptibility to oxidative stress damage of the skin due to polymorphism in genes that encode glutathione peroxidase 1 protein, and more particularly the polymorphism rs1050450, together with a genetic susceptibility to an appropriate elasticity of the skin due to polymorphism in genes that encodes matrix metalloproteinase-9 and matrix metalloproteinase-3, and more particularly the polymorphism rs3918242 and rs3025058 respectively.
Cluster 5_genetic susceptibility to oxidative stress damage of the skin due to polymorphism in genes that encode nuclear factor erythroid 2-related factor 2, and more particularly the polymorphism rs35652124.
Cluster 6_genetic susceptibility to loss of elasticity of the skin due to polymorphisms in genes that encode matrix metalloproteinase-3, and more particularly individuals with polymorphism rs3025058.
Cluster 7_genetic susceptibility to have an appropriate skin elasticity and antioxidant capacity due to polymorphisms in genes that encode matrix metalloproteinase-9, superoxide dismutase II and nuclear factor erythroid 2-related factor 2, and more particularly individuals with polymorphism rs3918242, rs4880, rs35652124 and rs6706649.
Cluster 8_genetic susceptibility to oxidative stress damage of the skin due to polymorphism in genes that encode nuclear factor erythroid 2-related factor 2, NAD(P)H dehydrogenase [quinone] 1 protein, glutathione peroxidase 1, and catalase, and more particularly individuals with polymorphism rs35652124, rs6706649, rs1800566, rs1050450 and rs1001179.
Cluster 9_genetic susceptibility to skin elasticity due to polymorphisms in genes that encode matrix metalloproteinase-9, and more particularly individuals with polymorphism rs3918242.
Cluster 10_genetic susceptibility against oxidative damage due to polymorphisms in genes that encode nuclear factor erythroid 2-related factor 2, superoxide dismutase II and catalase, and more particularly individuals with polymorphism rs35652124, rs6706649, rs4880, rs1001179, together with a genetic susceptibility to loss of elasticity and hydration of the skin due to polymorphisms in genes that encodes matrix metalloproteinase-9, interleukine-6, matrix metalloproteinase-1 and aquaporin-3, and more particularly individuals with rs3918242, rs1800795, rs1799750 and rs17553719.

### Algorithm 3: Algorithm to Improve Perceived Age Based on an Ensemble of Phenotypic and Genotypic Features of the Skin

The algorithm 3 of the present invention uses certain polymorphisms in one or more genes of an individual, in combination with certain physical properties of the skin, particularly its elasticity, hydration and antioxidant capacity, to predict the effect of one or a multiplicity of treatments in the perceived age of an individual.

The algorithm of the invention is based on a nonlinear transformation to project new points of each subject in the matrix x-genos and y-genos. System is trained by a Multilayer Perceptron (MLP) with a supervised backpropagation error to approximate the position of the genotype space from a phenotype vector.

Each treatment has a measurable effect on the three skin properties associated to skin oxidation, hydration and elasticity. The distance between each treatment with certain properties of the skin is defined. Particularly, a distance measure based on Hausdorff distance corrected by normalized Gaussian of sigma equal to 1 is defined and is selected the minimum distances between the targets of treatments and skin properties; hydration, oxidation and elasticity.

Each individual has a measurable genotypic based relation to the three skin properties associated to skin oxidation, hydration and elasticity. The distance between the proteins related to the individual genotype cluster profile (as described in algorithm 2) with certain properties of the skin is defined. Particularly, a distance measure based on Hausdorff distance corrected by normalized Gaussian of sigma equal to 1 is defined and is selected the minimum distances between the individual genotype cluster proteins and skin properties; hydration, oxidation and elasticity.

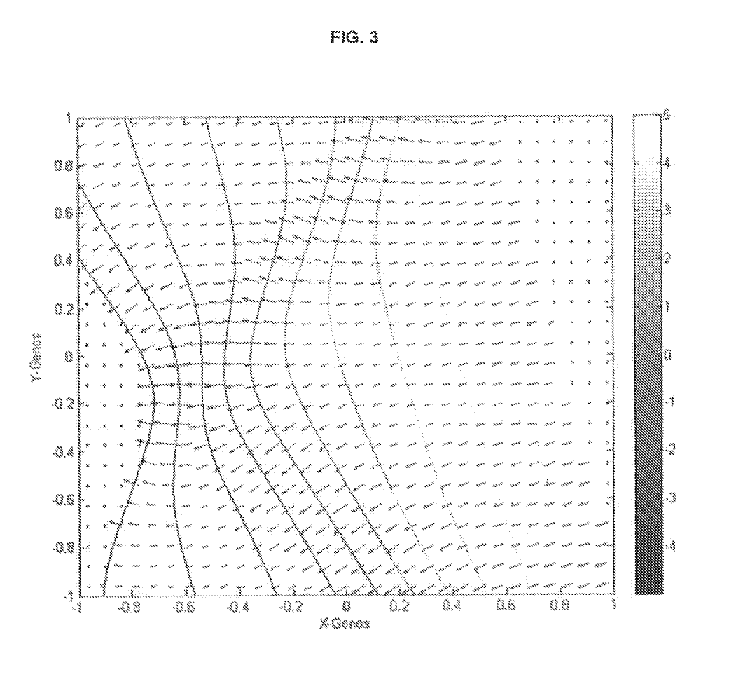

The algorithm uses machine learning techniques such as neural networks, or similar automated learning methodologies to learn how a treatment measurable effect on the three skin properties will change the x-genos y-genos position of the individual. In other words the algorithm learns to predict how a variation in the three skin properties maps to variation in x-genos y-genos (FIG. 3).

Particularly, the algorithm of the invention uses the information of the variation on measurable genotypic relation to the skin properties between each couple of individuals on a training cohort, and their variation in x-genos y-genos positions. The algorithm uses this information to learn what x-genos, y-genos variation will be caused by a treatment defined as a variation on measurable effect on the three skin properties.

The method predicts improvement on perceived age of the individual before and after the application of the treatment based on the x-genos y-genos positions of the individual before and after treatment as described in algorithm 1. The algorithm defines the optimal products and treatments as the ones that produce the greatest improvement of individual perceived age.

In a preferred embodiment, the algorithm is inserted into a system that comprises a computer-readable medium; at least one processor coupled with the computer-readable medium; and at least one human-readable output coupled with the computer readable medium and the processor system; wherein the system is capable of executing the algorithm based on an ensemble of phenotypic and genotypic variables to evaluate and improve the perceived age of an individual in a specified manner, comprising a database module creating and storing databases of biological data, a first unit operations module transforming the databases into physical and genotypic features, a second unit operations module generating at least one mathematical model, an analysis module executing experimental analysis and processes, and a comparison module comparing results arising from the models to at least a first set of empirical data.

The algorithm of the present invention is particularly useful to provide a diagnostic method based on determining a person's perceived age, useful to design an optimal treatment to improve person's perceived age.

More particularly, the algorithm of the present invention relates to a consistent and standardized diagnostic method that allows measurement of a perceived age over time, thus allowing validation of treatment which include but not limited to cosmetic treatment, exercise, nutritional complements, diets alternative medicine such as yoga, meditation, relaxation, pilates, laughter therapy, personal growth therapy, psychotherapy, and the similar.

The invention also relates to a method which determines a person's perceived age, useful to benchmark the product in order to determine its market value and customer claims.

The invention also provides an objective quantification of perceived age, which can be used as a measure to evaluate the overall health of one or more individuals, including but not limited to diseases associated to the key organs such as liver, lungs, kidney, heart, skin, muscles, bones and biological systems like the central nervous system, digestive and reproduction systems and the similar.

The invention also relates to a method useful to provide an objective quantification of perceived age, which can be used as a measure to evaluate the overall skin aging of one or more individuals.

### REFERENCES

1. Shekar, S. N., et al., Genetic and environmental influences on skin pattern deterioration. J Invest Dermatol, 2005. 125 (6): p. 1119-29.
2. Gunn, D. A., et al., Why some women look young for their age. PLoS One, 2009. 4(12): p. e8021.
3. Rexbye, H., et al., Influence of environmental factors on facial ageing. Age Ageing, 35(2): p. 110-5.
4. Christensen, K., et al., Perceived age as clinically useful biomarker of ageing: cohort study. BMJ, 2009. 339: p. b5262.
5. Taub, A. F., et al., Effect of multisyringe hyaluronic acid facial rejuvenation on perceived age. Dermatol Surg. 36(3): p. 322-8.
6. Chauhan, N., J. P., Warner, and P. A. Adamson, Perceived age change after aesthetic facial surgical procedures quantifying outcomes of aging face surgery. Arch Facial Plast Surg. 14(4): p. 258-62.
7. Guinot, C., et al., Relative contribution of intrinsic vs extrinsic factors to skin aging as determined by a validated skin age score. Arch Dermatol, 2002. 138(11): p. 1454-60.
8. Bulpitt, C. J., H. L. Markowe, and M. J. Shipley, Why do some people look older than they should? Postgrad Med J, 2001. 77(911): p. 578-81.
9. Oizumi, T., et al., A proposal for the cutoff point of waist circumference for the diagnosis of metabolic syndrome in the Japanese population. Circ J, 2006. 70(12): p. 1663.
10. de Koning, L., et al., Waist circumference and waist-to-hip ratio as predictors of cardiovascular events: meta-regression analysis of prospective studies. Eur Heart J, 28(7): p. 850-6.
11. Sparavigna, A., et al., Evaluation of anti-cellulite efficacy: a topical cosmetic treatment for cellulite blemishes—a multifuctional formulation. J Cosmet Sci. 62(3): p, 305-16.
12. Carruthers, A. and J. Carruthers, A validated facial grading scale: the future of facial ageing measurement tools? J Comet Laser Ther. 12(5): p. 235-41.
13. Bishop, C. M., Neural Networks for Pattern Recognition, ed, O. U. Press. 1995.
14. Sammon, J. W., A Nonlinear Mapping for Data Structure Analysis. Computers, IEEE Transactions on, 1969. C-18(5): p. 401-409.

1. A method for improving the perceived age of an individual that includes the steps of:
a. measuring phenotypic features of the skin;
b. measuring one or multiple polymorphisms in one or more genes associated with certain properties of the skin that contributes to perceived age;
c. inputting the phenotypic data into a computerized knowledge system that contains a first algorithm that predicts perceived age of an individual and that contains a second algorithm that classifies individuals into clusters defined as genotypic variables, associated to certain properties of the skin that contribute to person's perceived age, and that contains a third algorithm to define products or treatments to improve perceived age of an individual; and
d. determining the efficacy of products or treatments, and benchmark products or treatments in order to determine its market value and customer claims.

2. The method according to claim 1, wherein the algorithms are derived from an observed correlation between genotypic and physical variables and perceived age, and use a combination of the genotypic and physical variables to predict and improve the perceived age of individuals.

3. The method according to claim 2, where the phenotypic features of the skin are at least 2 features selected from the group consisting of wrinkles and roughness, number of wrinkles, depth of wrinkles, pigmentation, concentration of melanin, distribution (heterogeneity) of melanin, superficial vascular component, concentration of hemoglobin, distribution (heterogeneity) of hemoglobin, facial furrows, nasogenian furrow, labiomental groove, roughness, the position of eyebrows, state of periorbital wrinkles, state of facial wrinkles, evaluation of the labiomental groove.

4. The method according to claim 2, wherein the presence of at least one polymorphism is selected from the genes that encode the proteins matrix metalloproteinase-1, matrix metalloproteinase-3, matrix metalloproteinase-9, interleukin-6, aquaporin-3, NAD(P)H dehydrogenase [quinone] 1, superoxide dismutase [Mn], mitochondrial, nuclear factor erythroid 2-related factor 2, glutathione peroxidase 1 protein and catalase.

5. The method according to claim 2, wherein the polymorphisms are selected from the group consisting of rs1799750, rs3025058, rs1800795, rs3918242, rs17553719, rs1800566, rs4880, rs1141718, rs35652124, rs6706649, rs6721961, rs1050450, rs1001179 and combinations thereof.

6. The method according to claim 2, wherein the algorithms are neural network-based algorithms with an accuracy value of at least 92%.

7. The method according to claim 6, wherein the algorithms classify individuals by genotypic variables.

8. The method according to claim 7, wherein the genotypic variables are defined as a set of one or more polymorphisms of claim 4 that contributes to the perceived age of an individual.

9. The method according to claim 7, wherein the algorithms identify variations in the 22 variables that define the phenotype caused with each genotypic variable.

10. A method of evaluating the health status of an individual comprising using the method according to claim 1.

11. A method of assessing a therapy for an individual in need thereof, comprising using the method according to claim 1.

12. A method of assessing a pharmacological therapy for an individual in need thereof, comprising using the method according to claim 1.

13. A method of assessing a cosmetic therapy for an individual in need thereof, comprising using the method according to claim 1.

14. A method of assessing a cosmetic product for an individual in need thereof, comprising using the method according to claim 1.

15. A method of evaluating a treatment efficacy, that comprises the steps of:
a. evaluating initial perceived age of an individual using the method according to claim 1;
b. administering a treatment to the individual using the result of the method according to claim 1;
c. for each individual, determine a second perceived age value using the method according to claim 1;
d. for each individual, subtracting first value of perceived age from second value of perceived age; and
e. associating the difference with the efficacy of the treatment.

16. A system comprising:
a computer-readable medium;
at least one processor coupled with the computer-readable medium; and
at least one human-readable output coupled with the computer readable medium and the processor system;
wherein the system is capable of executing the method of claim 1 in a specified manner, comprising a database module creating and storing databases of biological, and phenotypic and genotypic data, a first unit operations module transforming the databases into physical features, a second unit operation module transforming the databases into genotypic features, a third unit operation executing at least one algorithm, an analysis module executing experimental analysis and processes, a comparison module comparing results arising from the analysis, and an optional output module providing automated interpreted results and assessings.
